(19) United States
(12) Patent Application Publication
(10) Pub. No.: US 2023/0321424 A1
(43) Pub. Date: Oct. 12, 2023

(54) INFUSION APPARATUS WITH INFECTION BARRIER

(71) Applicant: Michael Gabriel TAL, Tel Aviv (IL)

(72) Inventor: Michael Gabriel TAL, Tel Aviv (IL)

(21) Appl. No.: 18/077,847

(22) Filed: Dec. 8, 2022

Related U.S. Application Data
(60) Provisional application No. 63/287,817, filed on Dec. 9, 2021.

(51) Int. Cl. A61M 39/16; A61M 25/00

(52) U.S. Cl. CPC A61M 39/16; A61M 25/0026

(57) ABSTRACT

An infusion apparatus comprises a reservoir and a catheter having a proximal portion coupled to the reservoir. The apparatus comprises at least one antimicrobial barrier. The antimicrobial barrier may be configured to elute an antimicrobial agent into the surrounding area, such as into a fibrin sheath or clot on the external surface of the catheter.

### RELATED APPLICATIONS

This application claims priority to U.S. Provisional Application 63/287,817 filed on Dec. 9, 2021. The entire disclosures of all the related applications set forth in this section are hereby incorporated by reference in their entireties.

### BACKGROUND

Dialysis ports are totally implantable devices that are accessed through a needle for dialysis. The dialysis port includes a reservoir that is connected to a catheter. The catheter extends from the port through a subcutaneous tunnel and into a vein, usually the jugular vein. A fibrin sheath is always developing around the catheter, throughout its length. This fibrin sheath extends from within the vascular system, through the subcutaneous tunnel and connects directly to the capsule around the port and the space around the reservoir.

Dialysis ports are associated with extensive and fulminant infections associated with severe infections often leading to hospitalization and even death. These severe infections led to removal of these product from the market. Infections in ports, similarly to infections in any access catheter, can originate from two sources. Introduction of bacteria into the catheter hub during access. This is common in any access and especially with ports, as skin bacteria are always present, even after extensive cleaning. The needle used to access the port has to penetrate the skin on its way to the port, thus likely to introduce bacteria to the port pocket formed surgically in the body. The other source of infection is hematogenic, from the blood. Every patient, especially dialysis patient has transient bacteremia. Meaning bacteria circulating in the blood for a limited period of time, before the immune system eliminates it. With a foreign body in the vascular system such as a catheter connected to the port, there is a propensity of the bacteria to adhere to the catheter or any material on it such as clot or fibrin sheath. The bacteria can then grow and multiply until causing a systemic CRBSI (catheter related bloodstream infection).

The fibrin sheaths in ports extends from the port reservoir cavity (e.g., an inner space or cavity around the reservoir), along the catheter in the subcutaneous tunnel, and into the vascular system. In previous generations of dialysis ports, any infection at the port (reservoir) site would eventually extend into the vascular system, causing bacteremia, CRBSI and even sepsis and death. In addition, bloodstream infection related to an implanted catheter would extend to the reservoir area, thus reducing the effectiveness of antibiotic treatment and exacerbating the infectious process. This can cause a vicious circle of infection migrating from the reservoir to the circulatory system and vice versa, eventually culminating in an extensive infection event and sepsis. A phenomenon often seen in previous generations of subcutaneous dialysis ports, prompting cessation of their use, in spite of the clear clinical need for such a device in the End Stage Renal Disease (ESRD) patient population, especially those with poor access options.

Referring to the figures, one explanation for prior art dialysis port infection is illustrated in FIG. 1A showing a prior art port 20 implanted in a body of a patient and connected to a catheter 22. The port 20 includes an internal reservoir that can be accessed with needles through septums 21a and 21b. As shown, bacteria 28 are introduced to the area around the port 20 when the skin above the port is punctured for the purpose of access for dialysis. This access is performed three times a week. These bacteria multiply and because the capsule 26 around the port 20 is directly connected to the vascular system, the infection is likely to extend to the blood, making the patients bacteremic and sometimes also lead to hospitalization and death.

Another explanation for the higher infection rate in prior art dialysis ports is described in FIG. 1B, showing another prior art port 20 implanted in a body of a patient. As shown, recurrent bacteremia events can cause bacteria to multiply in the blood. This often leads to infection of the fibrin sheath 24. In dialysis ports, this bacteremia can lead to fulminant port pocket infection, which can further exacerbate the systemic bacteremia.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In one implementation An infusion apparatus comprises a reservoir, a catheter comprising a proximal portion coupled to the reservoir and a distal portion, and at least one antimicrobial barrier coupled to or integral with the catheter or the connection between the catheter and the reservoir.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIGS. 1A and 1B show prior art infected infusion apparatus.

FIG. 2 shows an infusion apparatus with a barrier.

FIG. 3 shows an infusion apparatus with a plurality of barriers.

FIG. 4 shows the infusion apparatus of FIG. 3 inhibiting migration of bacteria from the reservoir to the bloodstream of the patient.

FIGS. 5A, 5B, 5C, and 5D illustrate replacing portions of an infusion apparatus comprising a barrier.

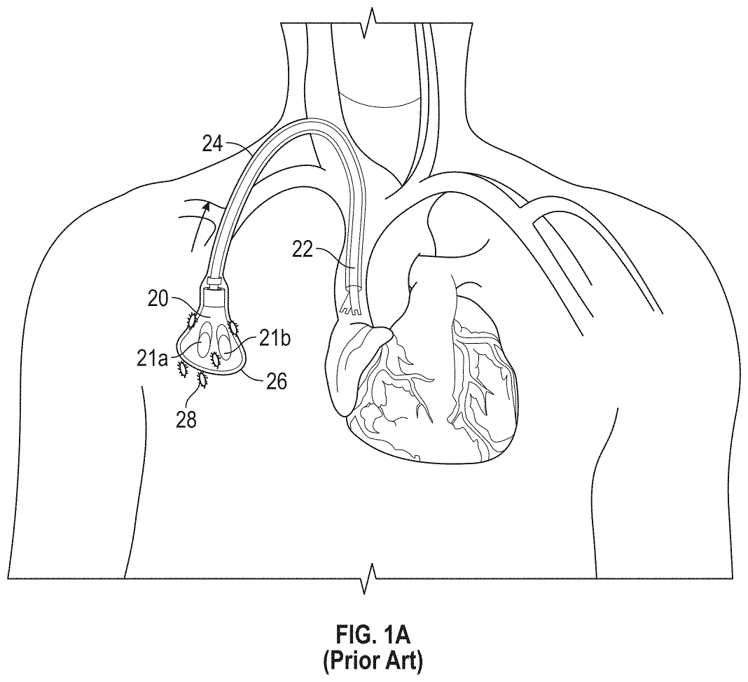

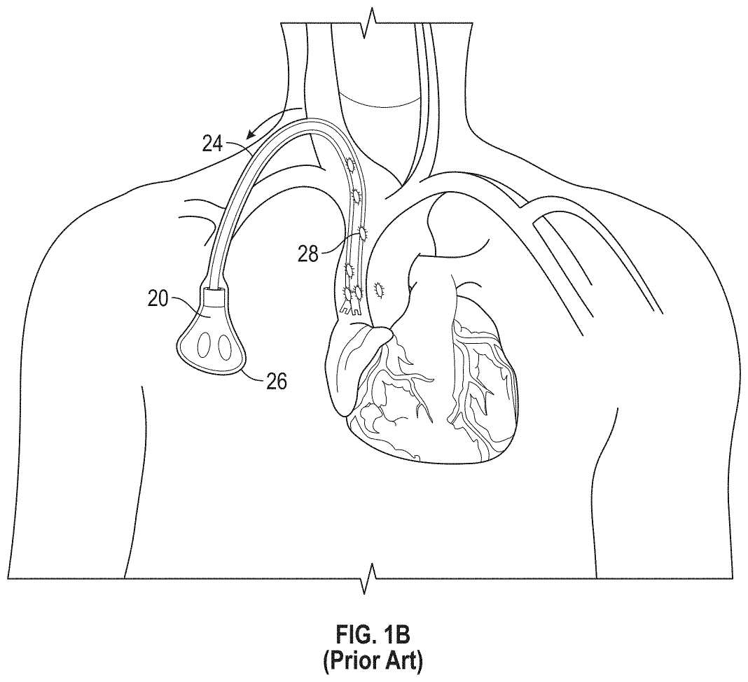

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Implementations of the technology described herein are directed generally to implantable infusion apparatus such as may be used for dialysis.

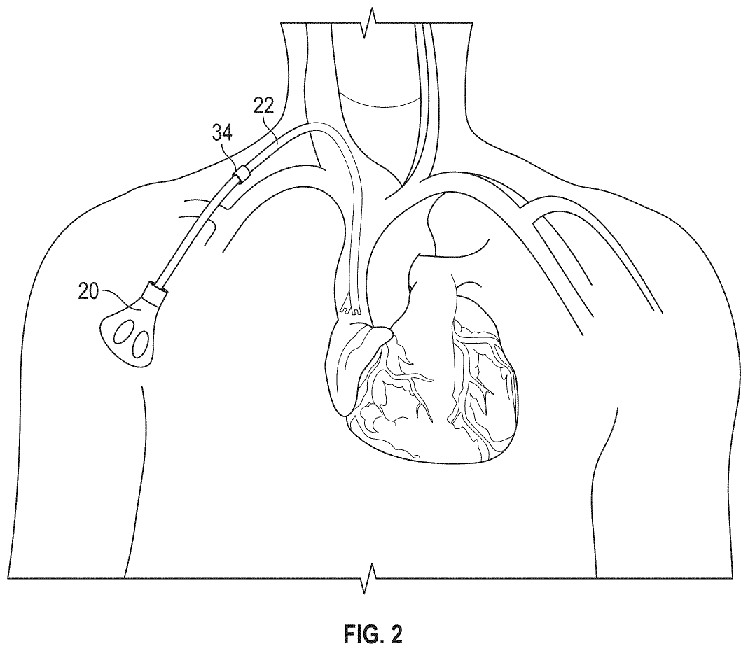

FIG. 2 schematically illustrates an exemplary embodiment of a port one or more barriers, according to some embodiments. The barrier 34 of FIG. 2 is configured to disrupt the advancement or continuity of the fibrin sheath between the port pocket 20 and the endovascular component 22. The barrier may be made for instance from Dacron, PET or other material. The barrier 34 may have an outer diameter greater than the outer diameter of the catheter 22 and may form a ledge or shoulder extending radially from the surface of the catheter 22 at its proximal and distal ends.

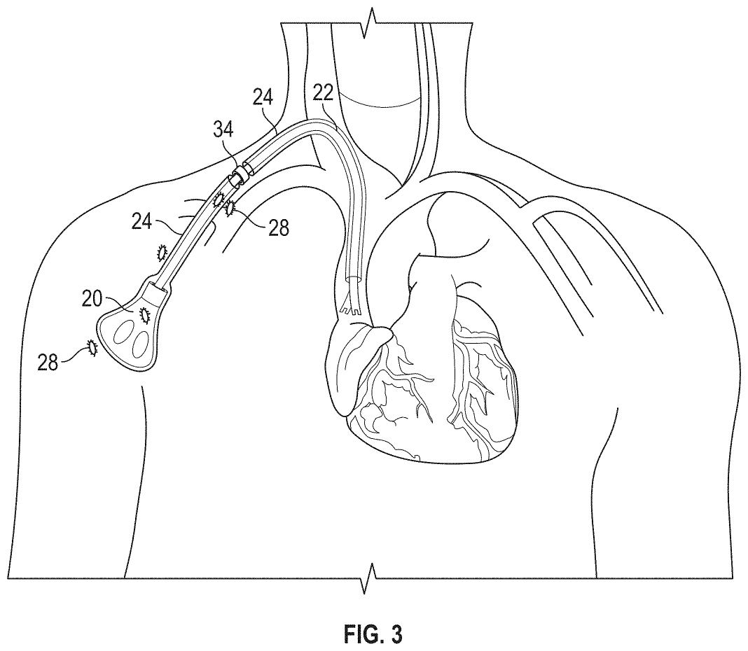

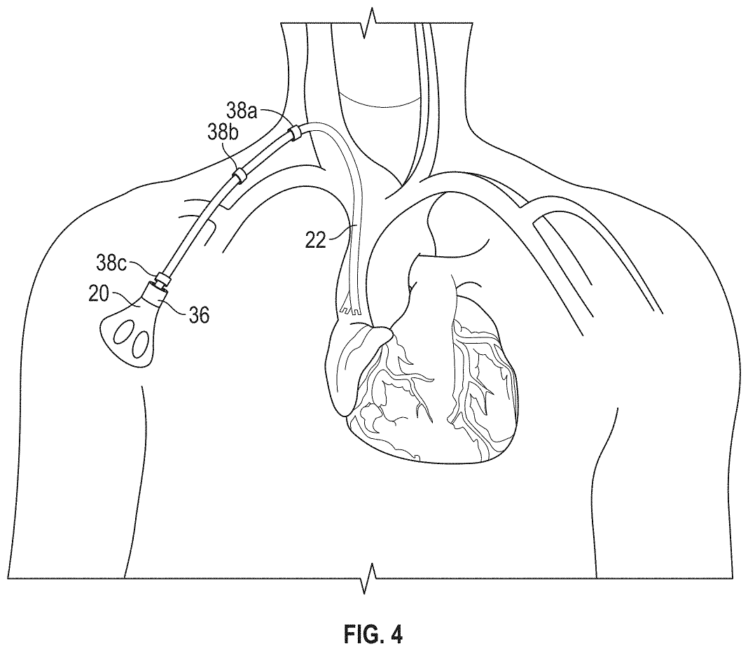

The barrier is optionally also configured to prevent bacteria present in the port pocket from passing therethrough for reaching the blood stream and causing bacteremia (CRBSI), as shown schematically in FIG. 3, according to some embodiments. As shown in FIG. 4, the fibrin sheath has a discontinuity at the barrier 34. It is optionally also configured to prevent bacteria from reaching the port pocket in the case of systemic bacteremia.

As shown in FIG. 4, according to some embodiments, a plurality of barriers 38a, 38b, and 38c which can be as described above with reference to barrier 34 can be placed at different locations along the extra-vascular part of the catheter or the connection between the catheter and the reservoir. The cuff/barrier can be placed next to or on the catheter lock 36 that holds the catheter tightly connected to the reservoir such as shown by barrier 38c of FIG. 3. The barrier(s) 34, 38 can have anti-bacterial propensities or properties, such as antibiotics or silver ion coating or another coating or material of any kind.

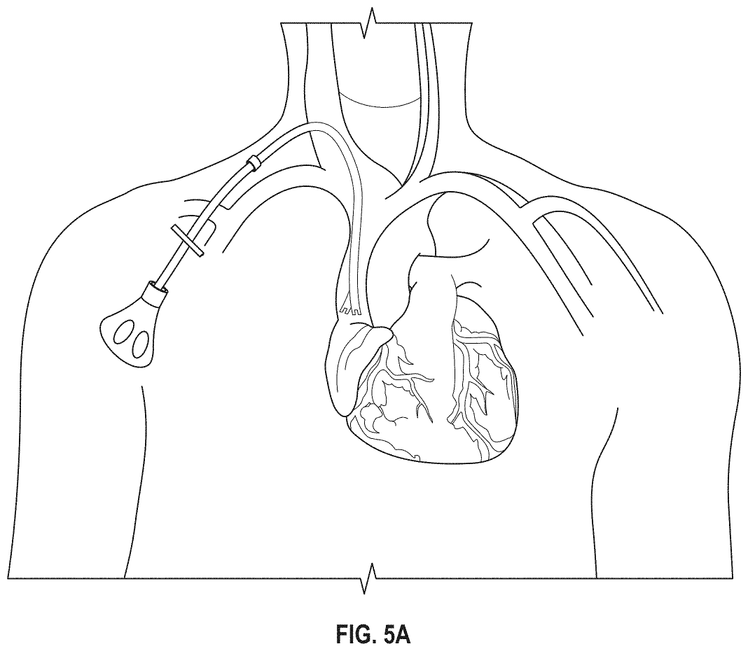

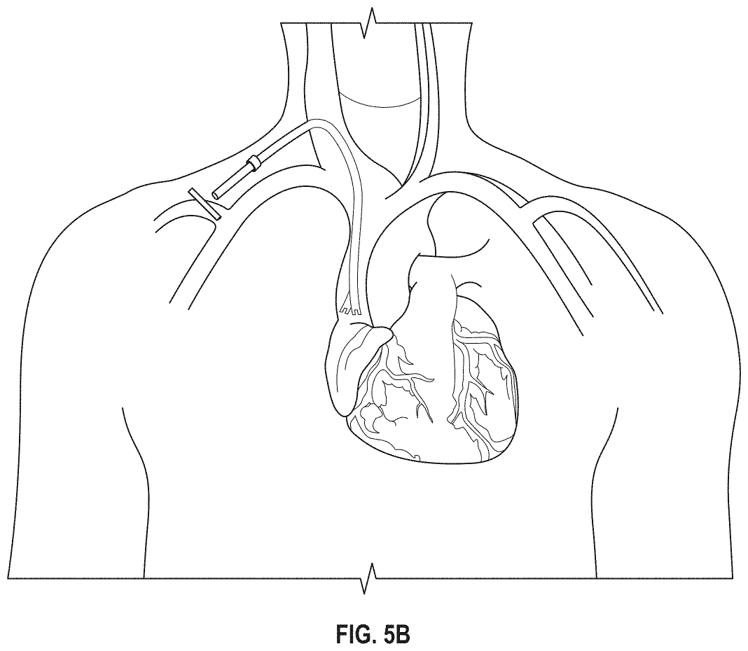

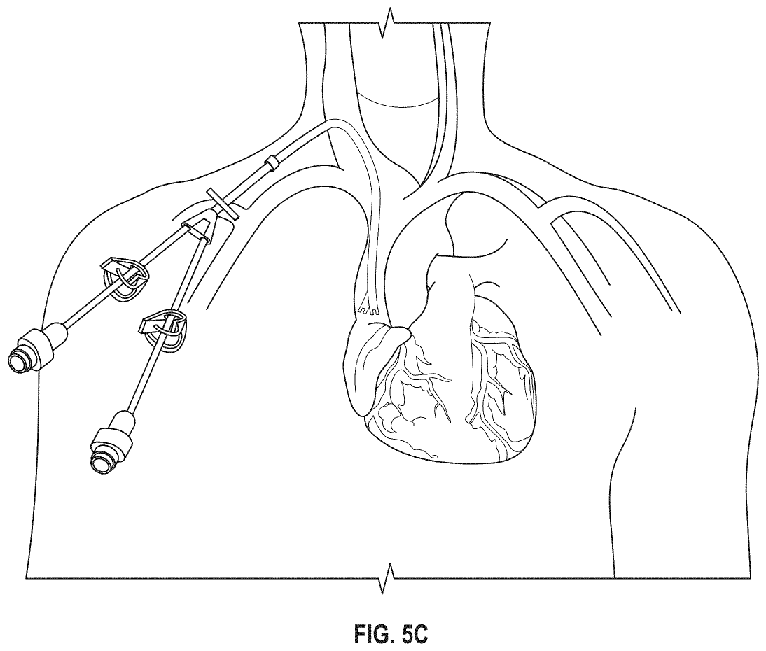

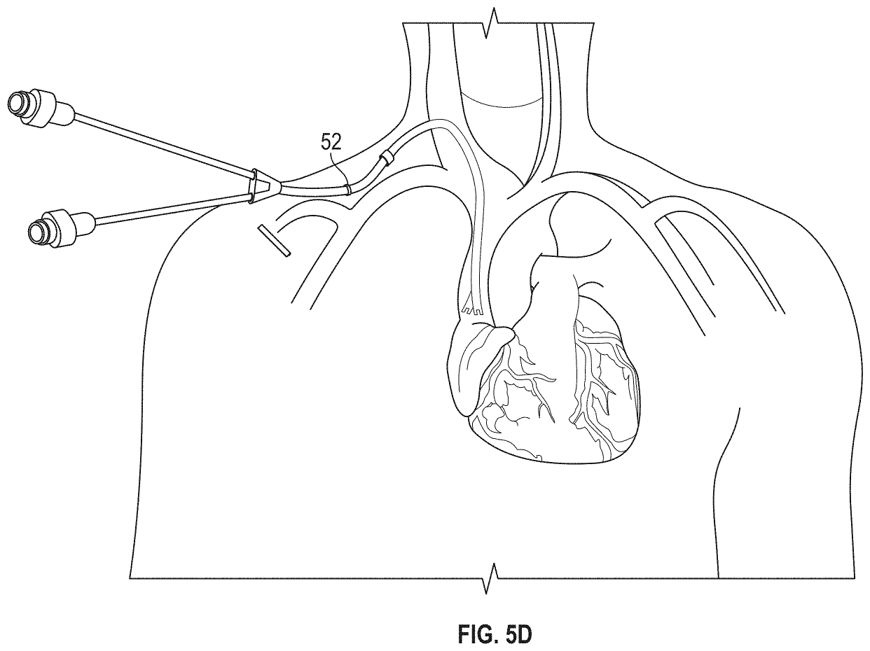

In cases of suspected pocket infection or systemic infection with non-cuffed ports, after a failed systemic antibiotic treatment, the whole port, including the reservoir and catheter, has to be removed. This leaves the patient with no access for dialysis. The only option is placement of another catheter at a different site. The dialysis patients that will be using hemodialysis ports are usually those lacking access sites and options. For these patients, a dialysis port infection and subsequent port removal can result in total, catastrophic, loss of access. This could also be one of the reasons that totally implantable dialysis ports failed in the marketplace. In some embodiments, and as schematically illustrated in FIGS. 5A, 5B, 5C and 5D, an exemplary microbial barrier may be configured to facilitate or allow salvage of the intra vascular catheter in case of port infection. In some such embodiments, the port includes special means (e.g., connector, coupling member, or cutting-ready portion) for allowing selective disconnection and/or removal of a previously implanted port reservoir and part of the catheter proximal to the barrier. The port reservoir and/or catheter portion can be replaced with new ones or with a different component or member, such as a Y-connector (shown in FIG. 5D, for example).

FIG. 5A shows port and proximal part of catheter to be removed through a skin incision. FIG. 5B shows catheter after removal of hemodialysis port reservoir.

FIG. 5C shows connecting the residual proximal catheter to a hub converting it to a regular retrograde catheter. The cuff/barrier can be placed on the catheter lock that holds the catheter tightly connected to the reservoir.

FIG. 5D shows a situation that the port pocket is grossly infected, thus the following exemplary method can be performed: making a small incision 52 in the skin away from the pocket and pulling the catheter through the incision and connecting it to a proximal catheter hub thus converting it to a regular retrograde catheter that is not inside the infected port pocket.

Optionally, after clearing the infection and healing of the port pocket a new reservoir can be placed and connected to the same catheter, thus preserving access.

As described herein, there is provided a device and method for preventing and reducing infections in totally implantable subcutaneous ports. In addition, a method for salvaging access in hemodialysis port patients with pocket infections, converting the infected port to a tunneled hemodialysis catheter, and optionally back to a subcutaneous hemodialysis port after clearing the infection.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. An infusion apparatus comprising:
a reservoir;
a catheter comprising a proximal portion coupled to the reservoir and a distal portion; and
at least one antimicrobial migration barrier coupled to or close to or integral with the catheter and/or the reservoir.

2. The infusion apparatus of claim 1, comprising a needle access port associated with the reservoir.

3. The infusion apparatus of claim 1, wherein the catheter comprises more than one lumen.

4. The infusion apparatus of claim 1, wherein the at least one antimicrobial barrier is configured to inhibit both proximal and distal migration of microbes along the catheter.

5. The infusion apparatus of claim 1, wherein the at least one antimicrobial barrier comprises an antimicrobial agent.

6. The infusion apparatus of claim 5, wherein the at least one antimicrobial barrier is configured to elute antimicrobial agent into the area surrounding the antimicrobial barrier.

7. The infusion apparatus of claim 1, wherein the at least one antimicrobial wherein the reservoir is detachable from the catheter.

8. The infusion apparatus of claim 1, wherein the at least one antimicrobial barrier is positioned to remain outside the vasculature of a patient during use.

9. The infusion apparatus of claim 1, wherein the barrier is placed on the catheter lock that holds the catheter tightly connected to the reservoir.

10. The infusion apparatus of claim 1, wherein the barrier is placed on reservoir adjacent to the catheter.
